(19) United States
(12) Patent Application Publication
(10) Pub. No.: US 2021/0369811 A1
(43) Pub. Date: Dec. 2, 2021

(54) Integrated COVID-19 Strategies

(71) Applicant: David M. Manyak, Burgess, VA (US)

(72) Inventor: David M. Manyak, Burgess, VA (US)

(21) Appl. No.: 17/332,683

(22) Filed: May 27, 2021

Related U.S. Application Data
(60) Provisional application No. 63/038,265, filed on Jun. 12, 2020.
(60) Provisional application No. 63/030,996, filed on May 28, 2020.

(51) Int. Cl. A61K 38/17; A61K 45/06

(52) U.S. Cl. CPC A61K 38/1732; C12Q 1/6827; A61K 45/06

(57) ABSTRACT

This disclosure provides for the application of a multi-disciplinary analysis of information sources to draw novel conclusions that result in new methods to diagnose, prevent or treat COVID-19. COVID-19 appears to be an extremely complex disease, encompassing three critical aspects at least: a viral infection, an immune system disorder, and a cardiovascular/pulmonary/renal disease with significant coagulation system dysregulation. This disclosure principally focuses on modes and sites of infection of the SARS COV-2 virus and the role of lectins, the lectin complement pathway, coagulation system dysfunction, related genetic polymorphisms, trypsin-like serine proteases, interferon stimulation of the innate immune system, and other factors in the disease process. Corresponding methods to address the COVID-19 pandemic are provided.

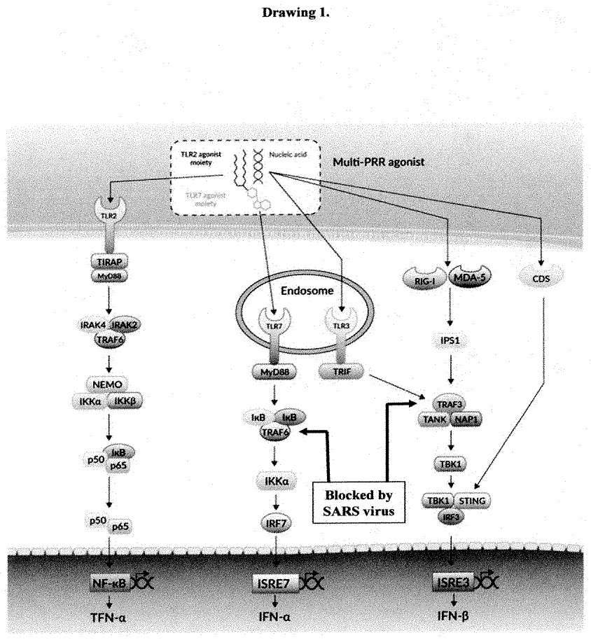

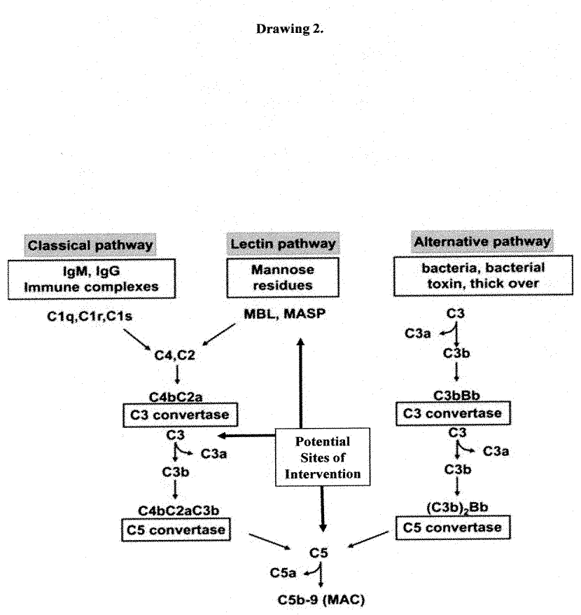

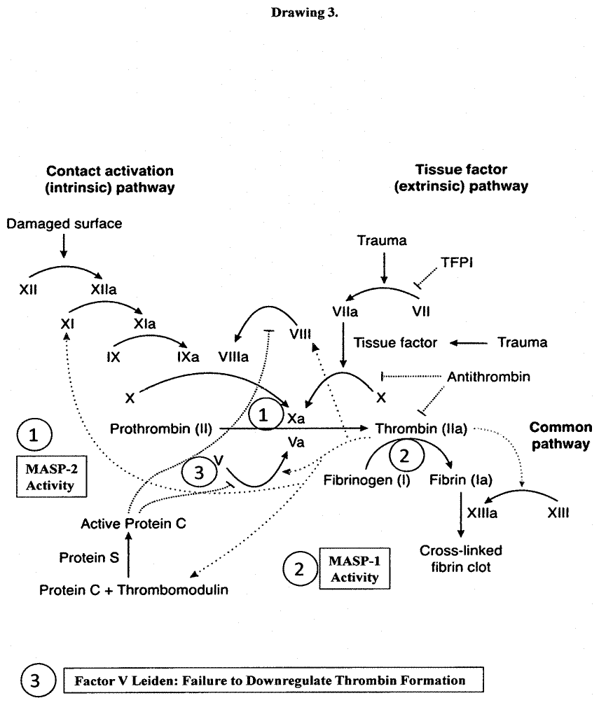

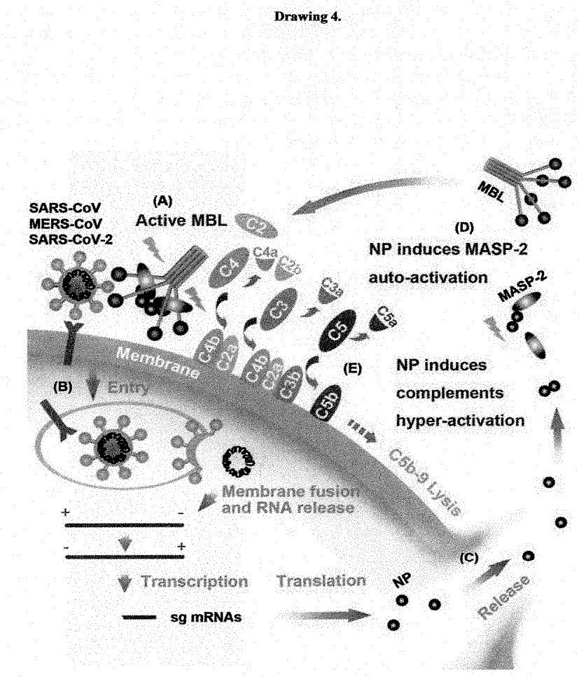

### CROSS REFERENCE TO RELATED APPLICATIONS. (RELATED APPLICATIONS MAY BE LISTED ON AN APPLICATION DATA SHEET, EITHER INSTEAD OF OR TOGETHER WITH BEING LISTED IN THE SPECIFICATION.)

This application claims the benefit of U.S. Provisional Application Ser. No. 63/030,996, filed May 28, 2020, and U.S. Provisional Application Ser. No. 63/038,265, filed Jun. 12, 2020. The entire disclosures of the provisional applications are relied upon and incorporated by reference herein.

### STATEMENT OF FEDERALLY SPONSORED RESEARCH OR DEVELOPMENT (IF ANY)

None.

### THE NAMES OF THE PARTIES TO A JOINT RESEARCH AGREEMENT IF THE CLAIMED INVENTION WAS MADE AS A RESULT OF ACTIVITIES WITHIN THE SCOPE OF A JOINT RESEARCH AGREEMENT

Not Applicable.

### REFERENCE TO A “SEQUENCE LISTING,” A TABLE, OR A COMPUTER PROGRAM LISTING APPENDIX SUBMITTED ON A COMPACT DISC AND AN INCORPORATION BY REFERENCE OF THE MATERIAL ON THE COMPACT DISC. THE TOTAL NUMBER OF COMPACT DISC INCLUDING DUPLICATES AND THE FILES ON EACH COMPACT DISC SHALL BE SPECIFIED

Not Applicable.

### BACKGROUND OF THE INVENTION

In late 2019, a widespread outbreak of viral infections initially centered in and around Wuhan, China, was identified. By March 2020, the outbreak had spread to a worldwide pandemic, affecting nearly every country and causing high morbidity and mortality. The infectious agent has been identified as a beta coronavirus (a single-stranded RNA virus), termed SARS COV-2, which is a novel infectious agent to which no general immunity had been established in human populations, and for which no available vaccine nor any known treatment for the resultant disease, termed COVID-19, existed. Therefore, there is a critical need for new unforeseen methods of diagnosis, prevention, mitigation, treatment, and cure to be discovered, developed, and applied. With the rapid rate of infection, time is of the essence for these critical needs.

Human coronaviruses were discovered in the 1960's. Relatively mild strains of coronavirus are thought to account for about a quarter of cases of the common cold. More deadly forms of coronavirus infections of humans emerged in the 2000's, notably SARS (2002-2004), which like COVID-19 was initially centered in China, and MERS (2012), centered in the Middle East. SARS in particular resulted in more than 8000 cases worldwide, with an approximately 10% mortality rate. A significant amount of scientific research was conducted related to SARS, but with limited follow-up to therapies or preventatives, in part because the disease spread stopped. No new cases of SARS have been reported since 2004. Nevertheless, the SARS research was important since the SARS coronavirus and SARS COV-2 share many similarities in the viral strains' genomes, biology, and modes of infection and virulence.

Although the general perception is that COVID-19 should be treated as a viral infection, and in particular a respiratory infection, many of the symptoms and morbidities exhibited by COVID-19 suggest a much broader range of effects than a simple respiratory infection. In fact, COVID-19 appears to be an extremely complex disease, encompassing three critical aspects at least: a viral infection, an immune system disorder, and a cardiovascular/pulmonary/renal disease with significant coagulation system dysregulation. Moreover, it may be that these aspects of COVID-19 are at least in part sequential, with the possibility that the viral “respiratory” infection is resolved relatively quickly in many patients (neutralizing antibodies and viral clearance are being reported as early as a week after symptoms of infection appear) while the adverse effects from dysfunction of the cardiovascular/pulmonary/renal and coagulation systems rage on in severe cases over many weeks. The latter are therefore important aspects of COVID-19 for therapeutic intervention.

The SARS COV-2 Virus is Reported to Attack Through the ACE2 Receptor

From a putative bat source and/or an intermediate animal host, coronaviruses have jumped to infect humans. For SARS and MERS, the person-to-person infectivity seemed to be relatively modest. Once the initial infection cycle ran its course, with the infected individuals building immunity and person-to-person transmission stopped, the SARS and MERS outbreaks ended. As noted, there have been no documented cases of SARS since 2004. With COVID-19, however, the person-to-person or potentially even surface-to-person transmission is much stronger. Even so, so far research has suggested that the mechanisms of infection are highly similar between SARS and SARS COV-2, but with the SARS COV-2 virus gaining a mutation or slightly modified genetic adaptation for enhanced functionality at the key virus binding and entry site on host cells, another set of mutations or genetic inserts that enhance the cleavage of a surface protein, or spike protein, of the SARS CoV-2 virus facilitating entry into host cells, and potentially gaining a greater evasive advantage with respect to the human immune defense system. Clearly these differences make SARS COV-2 more highly infectious and cause more far-ranging effects on human health vs. SARS.

The coronavirus that caused the SARS outbreak in 2002-04 has been shown to specifically bind to, and gain entry into human cells through internalization by, Angiotensin Converting Enzyme-2 (ACE2), a protease receptor found on the surface of many cell types. Recent studies indicate that SARS COV-2 infects human cells through the same ACE2 target as the SARS coronavirus. These coronaviruses contain external knobs (Spike proteins, which are specifically glycosylated) that can bind to the ACE2 receptor with high affinity. Once the SARS or SARS COV-2 virus binds to the ACE2 receptor, a second step is required for entry of the virus into the host cell. A protease, on the host cell surface, at least one of which has been identified as the trypsin-like serine protease called Transmembrane Serine Protease 2 (TMPRSS2), or perhaps a protease present in circulation or associated with a different cell type, cleaves a section of viral spike protein, which allows the remaining section of the spike protein to mediate fusion of the virus envelope membrane with the host cell membrane.

On SARS COV-2 Spike protein, there are two cleavage sites associated with this activation and fusion. One, the S1/S2 cleavage site, sometimes referred to as a “furin site”, has an insert of additional genetic sequences on SARS COV-2 compared with SARS. Based on modeling studies, this insert allows the site at which the cleaving serine protease (e.g., TMPRSS2, or furin) attaches and cuts to bulge out in a loop from the Spike protein. This loop, carrying a positive charge from two new arginine residues, presumably would facilitate attraction of a binding pocket of a serine protease with an embedded negative charge, such as an aspartic acid residue, increasing the effectiveness or probability of cleavage at the S1/S2 site. The result would be an increased host membrane fusing activity and therefore a more highly infectious SARS COV-2 virus. The second site, the S2 cleavage site, is also cut by a serine protease (again, such as TMPRSS2 as well), but appears to be basically the same structural sequence as the SARS virus S2 site.

Once these sites are cleaved, the virus is internalized into the host cell and can take over the machinery of that cell to replicate and make more viral copies. It does this using additional proteins that are part of the virus. Inside the cell, two large polyproteins are formed by translation of the viral RNA by the host cell's own synthetic organelles, and these polyproteins in turn are processed into the non-structural replicating proteins by two SARS COV-2 proteases, called 3C-like proteinase (3CLpro) and papain-like proteinase (PLpro). 3CLpro and PLpro are considered primary targets for protease-inhibitor antiviral drugs against SARS COV-2. Another protein that is formed is a replicase or polymerase used by the virus to make additional copies of RNA for packaging into new virus particles. This replicase is another target for antiviral drug development. Once more copies of the virus are made, the new viral particles are released into the bloodstream to infect additional cells. This process of release involves membrane fusion again between a vesicle inside the cell containing the new virus particles and the host cell membrane. Once released, the additional viral particles can infect other cells by the same or similar process. In addition, separate SARS COV-2 structural proteins such as nucleocapsid (N) protein may be released into the bloodstream.

The sequence of binding SARS CoV-2 to the ACE2 receptor and activation of the viral surface protein to allow cell fusion is not yet definitively known. The primary working hypothesis is that the virus binds to ACE2, then the activation of the viral fusion process occurs through the action of TMPRSS2 that is co-located with ACE2. Or can the virus be pre-activated for fusion somewhere else, then find an ACE2 receptor to bind to and start cell entry without further modification? Or are there alternative activating proteases other than TMPRSS2 and/or are there other molecular targets to which the SARS COV-2 virus can bind or be bound, either allowing entry into cells not expressing ACE2 or allowing proximity to the alternative activating proteases for pre-activation? These are important questions to help define the process of infection after exposure to the SARS COV-2 virus.

### BRIEF SUMMARY OF THE INVENTION

COVID-19, resulting from infection by the coronavirus SARS COV-2, is a multifaceted disease for which there is a critical need for new unforeseen methods of diagnosis, prevention, mitigation, treatment, and cure to be discovered, developed, and applied. COVID-19 appears to be an extremely complex disease, encompassing three critical aspects at least: a viral infection, an immune system disorder, and a cardiovascular/pulmonary/renal disease with significant coagulation system dysregulation. This invention relates to a multi-disciplinary analysis of information sources to draw novel conclusions comprising methods to diagnose, prevent or treat COVID-19.

### BRIEF DESCRIPTION OF THE DRAWINGS

Drawing 1.

Diagram of signaling pathways for innate immune response mediated by Toll-like Receptor-7 (TLR7) and RIG-1, showing components of pathways (TRAF3 and TRAF6) at which SARS-2 virus can block further signaling by which type 1 interferons would otherwise be stimulated to be expressed as an antiviral response to infection.

Drawing 2.

Diagram of Complement Pathways, from Noris and Remuzzi, (2013), especially the components of the Lectin complement pathway, showing potential sites of therapeutic intervention for treatment of COVID-19 by blocking the pathway at MBL/MASP (-1 and -2) (the preferred components to block) or Complement C3 or Complement C5.

Drawing 3.

Diagram of the Intrinsic and Extrinsic Coagulation pathways, showing sites (enzyme activity) at which (1) MASP-2 can promote the conversion of Prothrombin to Thrombin and (2) MASP-1 can promote the conversion of Fibrinogen to Fibrin, both of which can lead to formation of a fibrin clot, as well as (3) the site at which the polymorphism Factor V Leiden can lead to failure to downregulate the clot formation process by preventing Activated Protein C from converting Factor V to Factor Va, which in turn would otherwise limit further conversion of Prothrombin to Thrombin.

Drawing 4.

Diagram from Gao, T. et al. preprint 2020, “Highly pathogenic coronavirus N protein aggravates lung injury by MASP-2-mediated complement over-activation”, showing potential role of MBL and MASP-2 in potentiating SARS COV-2 replication via a positive feedback loop, and further suggesting use of inhibitors of MBL and/or MASP-2 for treating COVID-19.

### DETAILED DESCRIPTION OF THE INVENTION

### Summary of the Invention

COVID-19, resulting from infection by the coronavirus SARS COV-2, is a multifaceted disease for which there is a critical need for new unforeseen methods of diagnosis, prevention, mitigation, treatment, and cure to be discovered, developed, and applied. This invention relates to a multi-disciplinary analysis of information sources to draw novel conclusions about methods to diagnose, prevent or treat COVID-19.

This invention teaches that interferon alpha or interferon beta be administered to high risk individuals prophylactically and to early diagnosed patients with COVID-19, based on the analysis that bats, which are natural reservoirs for coronaviruses but show no ill effects, unlike humans, express type 1 interferon constitutively, and on the analysis that SARS COV-2 can inhibit the production of beneficial antiviral type 1 interferons in humans.

This invention teaches that lectins, especially those that bind mannose and/or N-acetylglucosamine, and in particular lectins such as banana lectin and griffithsin and derivatives thereof, are useful for diagnostic methods, prevention, and treatment of SARS COV-2 infection because of their high binding affinity for glycosylated proteins on the surfaces of viruses similar to those on the spike proteins of SARS COV-2 and their demonstrated ability to limit viral infection through such binding. Specific uses and methods are detailed in the Description below.

This invention teaches that, for SARS COV-2 that enters the gastrointestinal tract and potentially infects an individual through the intestines, consumption of a mannose lectin, preferably banana lectin in banana pulp, on a schedule and an amount sufficient to bind SARS COV-2 in a way to limit or prevent the virus' systemic entry through the intestines, is a potential treatment for COVID-19. Such consumption of banana may also allow banana lectin to enter the bloodstream through the intestines and bind to SARS COV-2 in such a way to limit infection by the virus into an individual's cells as an additional treatment modality for COVID-19.

This invention teaches that SARS COV-2 enters the gastrointestinal tract, that the small intestines are a site of potential systemic infection, and that some proportion of the virus will pass through the digestive system into feces, so that diagnostic tests based on fecal samples should be implemented and handling guidelines for items exposed to feces especially in high risk settings such as nursing homes need to be established for epidemiological control purposes. This invention also teaches that infectious SARS COV-2 is likely to be present in semen based on analysis of target and activating molecules in the testes and prostate, and that guidelines for abstaining from unprotected sex while individuals are infected should be established for epidemiological control purposes.

This invention teaches that the lectin complement pathway, including mannose-binding lectin (MBL) and its associated serine proteases, MASP-1 and MASP-2, play a central role in COVID-19, both positively in their innate immune system function and adversely, especially in severe cases in which the coagulation system becomes dysregulated and/or red blood cells are destroyed. This invention teaches that administration of inhibitors or blockers of MASP-1 and/or MASP-2 after the onset of infection is a preferred method of treating these adverse symptoms. This invention further teaches that inhibitors or blockers of components further down in the complement cascade such as complement C3 or complement C5 are also potential treatments for these adverse effects.

This invention teaches that dysregulation of the coagulation system evidenced in children and young adults with COVID-19, exhibiting symptoms being described as a Kawasaki Disease-like condition, is likely associated with genetic polymorphisms in one or more components of the coagulation cascade. In particular, individuals with Factor V Leiden mutation may be especially vulnerable to these adverse coagulation events, and genetic testing for Factor V Leiden should be implemented as a means to identify these at risk patients. This invention further teaches that inhibitors or blockers of MASP-1 and/or MASP-2 are potential treatments for this Kawasaki Disease-like syndrome associated with COVID-19.

This invention teaches that susceptibility to SARS COV-2 infection may be enhanced in individuals who have polymorphisms in the mbl2 gene that result in low or undetectable levels of the MBL protein. This invention teaches that an early diagnostic test following SARS COV-2 confirmation should be to measure circulating MBL levels in blood for evidence of depressed readings. This invention further teaches that screening for mbl2 genetic polymorphisms should be implemented to identify individuals with a heightened risk of SARS COV-2 infection.

This invention teaches the administration of inhibitors or blockers of trypsin-like serine proteases as a means of treating or preventing COVID-19 by limiting or preventing activation of the SARS COV-2 virus. Specific blockers may be plant-based serine protease inhibitors such as sunflower protease inhibitor (SFPI-1) that are administered orally including by consumption as a food. Another specific blocker may be an inhibitor or blocker of MASP-1 or MASP-2 administered systemically.

Other inventions should be apparent from the Description presented below.

### DESCRIPTION OF THE INVENTION

The Tissue Distribution of ACE2 Receptor Suggests Potential Sites of Entry and Infection

Given the above reported mechanism, it is important to understand where ACE2 is expressed as a protein in the body, in not yet infected individuals, and specifically where it is expressed at a site where there is potential exposure to the external environment, allowing transmission and infection from the external environment to the individual. These sites are most likely the sites of transmission of the virus from one person to another, or from viruses on surfaces into a person. According to a study from 2004 at the time of the SARS outbreak using more traditional histology methods (Hamming et al., 2004), the ACE2 protein, as one would expect from it being a key component of blood pressure regulation systems, is expressed on the epithelial cells lining blood vessels throughout the body, including within most organs. These sites, however, are all in general (except for tissue tears or open wounds) not exposed to the external environment. The two organs with extensive expression on surfaces affronting the external environment were the lungs (Type 1 and Type 2 alveolar cells) and the small intestines where ACE2 expression is concentrated in the brush border of the epithelial cell layer lining the small intestines, where the body takes up food nutrients. Therefore, these are two of the most likely sites of entry by the virus. Both the lungs and digestive system (e.g., small intestines) interface with the environment though the mouth and nasopharyngeal space. Importantly, Hamming et al. found high expression of ACE2 in the nasopharyngeal space but only on the basal (internal to the body) side, below the thick layer of squamous cells lining the mouth and nasopharyngeal space, suggesting that the mouth and throat may not be primary sites of viral attachment and entry into the body.

More recently (Ziegler et al., 2020), using single cell expression analysis, reported ACE2 RNA transcript expression in human nasal secretory (goblet) cells and ciliated cells in the mucosa of the upper airway and nasopharyngeal space. While these cells could represent additional and perhaps initial sites of infection by SARS COV-2, the percentages of such cells expressing ACE2 were relatively low (1-4% of these cell types). Furthermore, an even lower percentage of these cells (0.3%-1.6%) expressed both ACE2 plus the presumed activating enzyme TMPRSS2. While these cell types may well be initial sites of infection, their low density raises questions about whether this entry point would be sufficient to account for a widespread systemic infection. These cells, however, may represent the key sites at which components of the innate immune system in the mucosa (e.g., toll-like receptors) recognize and mount an initial response against the virus. Thus, low titers of virus exposure (but above a threshold required for a response) may induce an antiviral response at this level leading to asymptomatic cases of COVID-19 and eventual protective immunity. In this regard, Ziegler et al. (2020) also reported wide expression of genes that upregulate alpha interferon in these same cell types expressing ACE2.

Therefore, the most likely sites of infection are the small intestines, the oral/nasopharyngeal area, and the lungs. Most of the public advisories for COVID-19 prevention have focused on preventing transmission via airways (wearing masks, avoiding close exposure to aerosols from other individuals, etc.), yet ACE2 expression, based on classical histology, appears to be found in low levels in the upper respiratory tract, and the most likely movement of viruses that enter an individual through aerosol into the upper airway would tend to be back up into the oral/nasal area. The mouth, throat, nasal passages and upper airway tract are lined with the mucosa, a layer of mucus secreted by the endothelial cells that entraps particles that enter the nasopharyngeal space. Furthermore, these regions have extensive cilia on the endothelial cells that beat in a way to move the mucus up out of the airways and out of the nasal passages into the throat. There the mucus (with entrapped viral particles) is either expectorated or ingested down into the stomach. Similarly, saliva secreted into the mouth, which might also entrap or bind viruses, also is either spit out or passed down into the digestive tract.

Coupled with the fact that the worst areas of infections of the lungs with COVID-19 are deep in the lungs, not necessarily in the upper respiratory region, the above description would imply that a primary site of entry of, and systemic infection by, SARS COV-2 might be in the digestive system, not just the respiratory system, and rather that the lower lobes of the lungs may be infected in large part after the virus enters the body. In this case, SARS COV-2 would enter via ACE2 expressed on the Type II and Type I alveolar cells through circulation in the blood after primary infection elsewhere (although high levels of more deeply inhaled virus, or sustained inhalation or aspiration of virus particles over a longer period of time, could potentially enter deeper into the airways and directly infect the alveolar cells). This implication of a substantial role of infection through the digestive tract is a surprising conclusion given that the public responses to control of COVID-19 have been based on the assumption that it is a traditional respiratory infection. And if correct this analysis has a number of potential important implications for diagnostic testing, epidemiological control, and treatment of COVID-19, as discussed below.

The ACE2 expressed in the small intestines is densely located on the brush borders of the endothelial cells lining the small intestines. This location provides a ready interface to the external environment for viral infection. In addition to its primary function of regulating key functions in the renin-angiotensin system, ACE2 reportedly has a secondary function of transport of amino acids into the cell. Amino acid absorption from digested food is a primary function of the cells lining the small intestines. Thus, the presence of ACE2 in this cell layer could cause the virus to attach and be internalized, perhaps with activation by TMPRSS2, which is heavily co-expressed with ACE2 in these absorptive enterocytes, and subsequent membrane fusion, or perhaps even with the coincident function of food absorption. Furthermore, under some circumstances normally associated with inflammation, the small intestines exhibit a “leaky gut” syndrome, allowing larger molecules or complexes to leak into the bloodstream. These conditions seem amenable for SARS COV-2 to infect individuals through the small intestines.

Like the oral/nasal region, the surface of the small intestines has an extensive mucosal layer. Non-food particles are trapped in this mucus and carried down the remainder of the digestive tract to be excreted as feces. This may well include virus particles that have not been able to gain entry to infect the individual in the small intestines. Such a phenomenon would imply that SARS COV-2 might be excreted in feces. This has important implications for diagnostic testing and epidemiological control. Such a phenomenon also suggests a potential prophylactic or therapeutic strategy to reduce viral titers infecting the individuals at the time of exposure or soon thereafter.

The following are potential important implications of the above analysis on viral entry sites:
1. To date, the focus of diagnostic testing for SARS COV-2 infection has been on taking nasopharyngeal swabs to use for PCR-based viral RNA detection. If SARS COV-2 is only transiently present in the oral/nasal/upper airway region where the swab is taken because there is not a significant active infection (i.e., not a significant source of ACE2 exposed to the surfaces in this area), then one might expect to have false negatives for detecting infection from this approach, and/or cases where some tests in the same individual are positive and some are negative at different times with the same individual. The same mix of false negatives may occur simply from the scattered distribution of the relatively few goblet and ciliary cells expressing ACE2 at which the virus could infect vs. the location from which the swap is taken. At the least, it might suggest that this diagnostic method is not a reliable early detection method. After a systemic infection occurs, then the oral/nasal/airway may be more highly infected from the infection of endothelial cells in this area that contain ACE2 on the basal side (i.e., toward the bloodstream), in which case the test would more likely detect the presence of virus as these cells become more infected.
2. The likelihood that virus passes through the digestive tract suggests that a diagnostic test using stool samples would be extremely important to deploy, and it is surprising that this approach has not seemed to be part of the public response to COVID-19. Tests for SARS COV-2 using stool samples have been reported in some research settings and have detected SARS COV-2 virus, or at least viral RNA. Whether live virus is present in the samples has apparently not yet been demonstrated, but it is likely. In one study of 131 patients in China (Chen, et al., 2020), positive PCR tests for SARS COV-2 were found in 22 patients in fecal and sputum samples for a period of up to 13 and 39 days respectively after the patient's tests with nasopharyngeal swabs had turned negative. In a case study of an Australian COVID-19 patient, the initial positive (on hospitalization) nasopharyngeal test was followed the next day with a positive fecal test. It takes approximately 4-6 hours for food to reach and pass through the small intestines after ingestion, and 24-44 hours for food remnants to be excreted. So this sampling approach would not be an immediate indication of viral infection, but it may be more reliable if the testing occurred every day especially for high risk individuals such as health care workers or residents of nursing homes. Such testing may provide an indication of exposure to the virus with a 1-2 day lag.
3. Most surprising is that precautions regarding disinfection of surfaces exposed to feces has not been part of the public advisories for preventing infectious spread, if nothing more than a precaution just in case such spread could occur. In particular, one might wonder if the severe spread seen in outbreaks in nursing homes, health care settings, and prisons might be due at least in part to inadequate handling of bedpans, beddings, toilet facilities, and other possible reservoirs for fecal material.
4. Another source for diagnostic testing could be saliva, especially if the sampling is done over a period of time to allow a more widely distributed sample of the oral/nasopharyngeal space and a higher extent of capture of virus to enable a more accurate test. Testing based on sputum could also be a good indicator of deeper lung or upper airway infection but may not be a good test for early diagnosis of SARS COV-2 infection. A potential diagnostic test for systemic or more severe SARS COV-2 infection may be based on use of blood samples, although this testing method would not capture the earliest stages of infection. To date, surprisingly, such a test has apparently not been deployed. A blood-based test would most likely correlate with severity of infection and be important to monitor the rate of clearance of SARS COV-2 infection and the efficacy of antiviral treatments. The current paradigm of nasopharyngeal-based tests, being more scattered or transient in their detection of virus as noted, is unlikely to be useful for such later stage infection monitoring.
5. A diagnostic test based on a virus capture method in the mouth and/or nose, or capture on a mask worn over the mouth and nose, could be the best option for detecting early exposure. In this approach, a much longer exposure time to the virus could be achieved compared with a nasal swab, maximizing the chance of capturing enough viral particles for accurate diagnoses. One possibility for an oral test may be use of a chewing gum or other generally accepted as safe substrate that contains a moiety that binds the SARS COV-2 virus, such as a lectin, preferably a mannose-binding lectin such as banana lectin or Griffithsin or similar molecules, or antibodies against SARS COV-2 surface proteins. In this case, chewing the gum or other substrate for a length of time, perhaps a couple hours, might capture enough virus particles to enable an accurate diagnostic test. This method of detection would also preferably rely on a means to release the captured virus from the substrate after removal from the mouth during the laboratory measurement step. There are numerous examples among products in the life sciences industry in which a capture molecule is bound to a substrate, such as beads in a column or a reactive surface, through a linker to a capture molecule to perform, for example, affinity chromatography. [A representative example is ThermoFisher product 20395, an agarose bead for column chromatography containing bound jacalin (a galactose targeting plant lectin) used to capture secretory IgA. Jacalin could be replaced by a mannose-binding lectin for SARS COV-2, or tested for its ability to bind SARS COV-2.] The captured molecule is then eluted off of the bead or surface by, for example, dissolving or breaking the linker substance. Once eluted and concentrated, a PCR-based or ELISA-type assay could be performed. Another possibility might be the use of chewing tobacco as the oral capture method in which the tobacco plant has been genetically engineered to produce a capture molecule. For example, recombinant Griffithsin, an algae-derived mannose binding lectin with strong affinity for SARS virus, has been manufactured using field-grown tobacco plants (Fuqua et al., 2015, Alam et al., 2018).
6. Biomarker tests for indicators of early infection by SARS-COV-2, rather than detection of SARS COV-2 itself, could also be valuable tools for relatively early detection of COVID-19.

Another site of high levels of expressed ACE2 receptor is the testes. ACE2 is also expressed in the kidney, brain, and heart, or on endothelial cells of blood vessels in these organs, but none of these sites are exposed to the external environment. Nevertheless, once an individual is infected, and the virus enters the blood system, these are all sites for potential cellular infection and adverse effects from alteration to the normal function of the infected cells. Of particular concern that has not been widely described is the presence of ACE2 expression in the testes, coupled with the known presence of TMPRSS2, as noted an enzyme that is reported to be needed to activate and prime SARS COV-2 for cell fusion and infection, in the prostate. A report from China (Li et al., 2020) indicated that of 38 hospitalized male COVID-19 patients enrolled in a study, 6 had positive diagnoses by PCR in samples of their semen, 2 of whom were in the recovery stage of their infection. TMPRSS2 has been implicated as a marker for prostate cancer, and is reportedly upregulated in response to male hormones (androgens) (Lin et al., 1999). Its normal role in the prostate appears to be processing semen by protease action to make it more fluid. The presence of both ACE2 and TMPRSS2 in the male reproductive organs should raise concerns about whether activated SARS Cov-2 can be sexually transmitted. This could be another important epidemiological control of refraining from unprotected sexual activity while actively infected. Furthermore, this additional reservoir for viral infections, and reported upregulation of TMPRSS2 in response to androgens, may contribute to the observed greater incidence of severe COVID-19 infection in males vs. females.

SARS and SARS COV-2 Coronaviruses Likely Came from Bats

The SARS and SARS COV-2 coronaviruses are thought to be zoonotic (animal-derived), existing in animal reservoirs and being passed to humans from these animals, causing human infections. The putative primary hosts of these two coronaviruses are bats. Bat species have traditionally been divided into megabats (or fruit bats) and microbats (mostly echo-locating insect-eaters), but more recently they have been reclassified (in part on genomic data) into the suborders Yangochiropiera and Yinpterochiroptera, the latter of which include the fruit bats and some of the microbats, including the horseshoe bats. Bats have been shown to be major carriers of a range of viral infections that have affected humans and animals, including important food animals. For example, fruit bats are believed to be the source of the Marburg filovirus outbreak in 2007 and to be a reservoir for Ebola virus. Both certain fruit bats and the related microbats carry Nipah and Hendra viruses. The MERS virus is believed to be from bats, transmitted through camels. The putative source of the SARS coronavirus is a horseshoe bat native to China, possibly transmitted from the bats to humans via a civet cat. And the SARS COV-2 virus is also thought to be derived from bats, possibly through pangolins or snakes as intermediate hosts. This origin for SARS COV-2 is still not definitively known, and genomic analysis has suggested some significant additions or changes in their RNA sequence (i.e., the significant insert at the S1/S2 cleavage site, or furin site) compared with other known coronaviruses, raising questions of an artificially introduced genetic component, or a genetic recombination with another source at some point in time. Nevertheless, bats are known to be major reservoirs of coronaviruses, and SARS COV-2 likely had its origins in some form from bats.

Why Don't Bats Get Symptoms of Viral Infection?

Although bats are carriers of these many viruses, including the coronaviruses, they do not show signs of infection, yet the same viruses are highly infectious to other mammals including humans when transmitted. Why is that? The first possibility is that there are special adaptations of the bat immune system, especially the innate immune system. Zhou et al (2015) has reported that in the Australian flying fox bat Pteropus alecto, the genome is highly compressed with respect to alpha interferon (IFN-a) genes (only 3 genes) vs. other mammals that typically have 7-18 IFN-a genes, and that unlike other mammals, IFN-a is constitutively expressed in the bat's unstimulated tissues. In humans, IFN-a (i.e., type 1 interferons, which includes IFN-a and IFN-beta) is stimulated to be expressed in response to detection of viruses by the innate immune system, but is not constitutively present. The always-on anti-viral activity of IFN-a in bats may be an adaptation to provide resistance to viral infections and circumvent the strategies that some viruses (including coronaviruses) have to inhibit or disable the innate immunity signaling pathways that ultimately would otherwise lead to IFN-a expression and downstream antiviral responses. There is evidence that Toll-Like Receptor 7 (TLR-7, innate immune system component, as discussed below) signaling is inhibited in humans by coronaviruses, potentially blocking the pathway to transcriptional activation (via interferon response elements, or IREs) of genes to produce type 1 interferons.

One conclusion from analysis of the difference between bat and human innate immune adaptations in bats vs. humans is that administration of alpha interferon or beta interferon, both of which are drugs in routine clinical use worldwide for other therapeutic indications, may be an effective treatment for COVID-19. Since in bats type 1 interferons are constitutively expressed, by analogy, this would suggest that administering alpha interferon or beta interferon to humans would best be done either prophylactically to high risk individuals or at the time of exposure to the virus or on initial infection. This would require the availability of a diagnostic screening test or method that would detect the SARS COV-2 virus rapidly after exposure or initial infection, or a decision and policy to administer IFN alpha and/or IFN beta to high risk individuals prophylactically.

One potential caution in implementing this strategy is that the ACE2 gene has been reported to contain an upstream gene sequence that binds STAT1, a transcription factor stimulated in response to alpha interferon, suggesting that ACE2 expression may be upregulated by interferon. Ziegler et al. (2020) found evidence that alpha interferon stimulated increased expression of ACE2 in certain nasal secretory cells. In this case, interferon may increase the number of ACE2 targets available for viral attachment and infection. Whether this phenomenon leads to actual increases in ACE2 protein on key cell surfaces in COVID-19 infection or has an impact on the course of infection remains to be seen and even so may only come into play on more severe infections with more virus being present at the time of new synthesis of ACE2 protein. Furthermore, additional synthesis of ACE2 receptor during active infection may have a beneficial role in that internalization of ACE2 receptor by SARS COV-2 attachment and internalization reduces the amount of ACE2 present to perform its role in the renin-angiotensin system. ACE2 is critical for reversing the vasoconstrictive effect of angiotensin II in the bloodstream.

A second adaptation or ecological consequence for controlling virus infection symptoms, at least by fruit bats, may be related to their diets. Fruit bats eat bananas, mangoes, figs, avocados, and dates, in particular. Of these, all but dates (and they may too) are reported to contain significant levels of lectins, which are proteins that bind to specific sugar residues of glycoproteins. Lectins are generally resistant to stomach acids and digestive processes so they mostly remain in the digestive tract. Viruses, including coronaviruses, have surface proteins that are decorated with various sugar residues at the external most projections. Numerous plant lectins have been shown to specifically bind with patterns of sugar residues on virus coats. One possible consequence of the diet of fruit bats being high in lectins is that the lectins can bind up viruses in the gut and sequester them to be passed out in feces, limiting the amount of virus able to infect the host bat through entry in the small intestines. Some lectins can also pass through the tight junctions in the small intestines (leaky gut) and enter the bloodstream where they can also bind up viruses to limit infectivity toward the host cells.

For coronaviruses, the key external surface glycoprotein is the spike protein, which radiates out from the virus core in large numbers, giving coronaviruses their characteristic “corona” appearance. Although the putative source for the SARS and SARS COV-2 viruses is a horseshoe bat (i.e., not a fruit bat, but an insect eater), it has been demonstrated that a number of purified mannose-binding lectins, especially banana lectin, bind with high affinity to the SARS virus' spike protein, and in doing so are able to inhibit the virus in lab studies, as described further below. Insects contain lectins as well, possibly from eating lectin-containing plants. Lectins are considered an elementary form of innate immune defense used by plants and lower animals, and by mammals as well.

One novel conclusion from analysis of the diets of bats with respect to possible means to limit viral infectivity, may be to administer certain fruits or other foods rich in lectins, or other preparations containing such food-derived lectins, or purified or recombinant lectins by oral route as an effective treatment for COVID-19. In particular, for example, without limiting other options, consumption of bananas may be one preferred method for treatment of COVID-19.

Returning to the above discussion of a primary route of SARS COV-2 infections in humans potentially being the small intestines, consuming food containing molecules that can bind the virus in such a way that it cannot gain entry into the cells lining the small intestines could allow the immobilized virus to pass through the digestive tract, thus preventing or limiting the degree of infection. Preferably these binding molecules would have characteristics that allow them to survive passage through the stomach intact, be active in the lumen of the intestines, and be large enough (high molecular weight) not to pass through the gut into the bloodstream. Furthermore, preferably the lectins should be in sufficiently high concentration in the food material, and the active lectin itself should have a relatively high binding affinity for the SARS COV-2 virus. And further preferably, the binding should be to a portion of the SARS COV-2 virus, such as the spike protein, so that the virus's affinity to its infectious target (e.g., ACE2) or its ability to fuse with the endothelial cells of the small intestines is limited or prevented, either directly or sterically.

Two lectins in particular that bind to mannose sugars on surface glycoproteins of viruses and have been shown to possess antiviral activity are banana lectin (aka, BanLec) from bananas and plantains, and griffithsin, a lectin found in red seaweeds (Griffithsia sp). Another less relevantly documented but potential anti-viral lectin is cyanovirin-N from Nostoc species. Many additional lectins, especially lectins directed toward mannose residues on proteins, may be useful (which would need to be confirmed with respect to their binding to SARS COV-2), including but not limited to the following: leek lectin, mango lectin, pineapple lectin, lectins from other algae genera or species (such as Porphyra, Palmeria, Agardhiella, Gracilaria, etc.), soybean lectin, garlic lectin, snowdrop lectin, amaryllis lectin, lentil lectin, jacalin (lectin from jackfruit), etc. Some of these lectin sources may not be suitable for consumption without further safety studies although many of them are contained in widely consumed food sources.

Bananas, containing banana lectin, may represent an especially promising possibility for limiting infection of SARS COV-2 through the gut, both because banana is a widely consumed food and because its lectin has relevant attractive biochemical properties. Banana lectin is a dimeric protein, with each subunit having a molecular weight of 15 kD, and has a high affinity for binding to mannose and mannose containing glycoproteins. It is found at relatively high levels in the pulp of bananas (about 4 mg/100 g of pulp, with an average banana being about 100 g). Purified or recombinant banana lectin (or BanLec) has been shown to bind to the glycosylated surface protein GP120 of human immunodeficiency virus (HIV) (Swanson et al, 2010), and in doing so to strongly inhibit HIV viral entry into cells in culture. The effect is very potent, with an IC-50 value in the low nanomolar to picomolar range. Hopper et al. (2017) modeled BanLec binding mechanisms with HIV and found BanLec assembled into tetramers with multiple binding sites on GP120, leading to aggregation of virus protein and again blockage of viral entry. Coves-Datson et al. (2019) demonstrated that a recombinant variant of BanLec (H84T) inhibited the Ebola virus by similar mechanisms. Furthermore, the BanLec variant was administered to mice (intraperitoneally) that were challenged with an otherwise lethal dose of Ebola. Partial protection (50-80% survival) was achieved in the treated mice, including mice that were pretreated before challenge. Keyaerts et al. (2007) tested 33 plant lectins (but not BanLec) in a SARS virus infectivity test in vitro and found 10 lectins that were inhibitory, with leek (mannose-targeted) lectin having the most potency of high nanomolar EC-50. Similar to HIV and BanLec, the mechanism identified was binding to the coronavirus envelope (Spike) protein and preventing entry into the cell, as well as limiting exit of new virus particles from the host cell. These datapoints suggest banana lectin, and potentially other mannose binding lectins, could have an inhibitory effect on SARS COV-2.

Some potential routes of administration and doses of banana lectin could be to consume one medium sized banana each in the morning, noontime, and evening. Since it takes 4-6 hours for food to clear the small intestines, this dosing could potentially provide for nearly continuous presence of banana lectin in the small intestines during the daytime, the period of likely virus exposure. Because of the fairly high levels of banana lectin in a banana, one banana may theoretically be an effective dose. For example, 4 mg of banana lectin (per banana) with a MW of the dimer of 30,000 would represent 1.33×10⁻⁷ moles. Dividing by Avogadro's number yields about 10¹⁶ molecules equivalent. An average coronavirus particle has 74 spike proteins (Wiki), which at a 1:1 binding stoichiometry with banana lectin (it is potentially 2:1 or 3:1 lectin per spike protein) would suggest the potential for one banana to bind about 10¹⁴ virus particles. Even if these input numbers or calculations are incorrect, the relative magnitude suggests a significant binding capacity per banana. Clinical trial experimentation by one ordinarily skilled in the art would help define an optimal consumption schedule and amount for effective dosing.

Human Lectin Pathway in Innate Immunity

Humans also use lectins as one component of their defenses against infectious agents. The innate immune system is designed to recognize foreign pathogens and form a first line of defense by the body in holding that pathogen at bay until the adaptive immune system can form a more permanent defense. This process eventually results in the production of neutralizing antibodies by the body (adaptive immune response) which can specifically identify, bind to, and mediate clearance of the targeted invading pathogen, in this case the SARS COV-2 virus. However, the first production of antibodies through the adaptive immune response generally doesn't occur until about one to two weeks after initial infection by a new pathogen naïve to an individual. Unfortunately, severe cases of COVID-19 can progress more rapidly to adverse outcomes, morbidity or death, than the timeframe for antibody appearance. A critical goal for mitigating COVID-19 should therefore be to control viral levels and viral infectivity until neutralizing antibodies can form and clear the infection. This should be the role of an effectively functioning innate immune system.

The innate immune system depends first on a pattern recognition receptor system to identify molecular or chemical structures (Pathogen Associated Molecular Patterns, or PAMPs) that are foreign to the body; e.g, only found in pathogens. One main system for pattern recognition is a series of toll-like receptors (TLRs) expressed on or in certain immune system cells, especially dendritic cells and macrophages. In humans, there are 10 different TLRs known, each having a specific type of molecular structure that they recognize. Two of these TLRs are TLR7 and TLR8. Both of these target single-stranded RNA. The SARS COV-2 virus is a single-stranded RNA (ss-RNA) virus. Therefore, recognition of SARS COV-2 by TLR7 in particular should initiate an innate immune response against the virus. There is evidence from studies on SARS coronavirus that this process involving TLR7 is indeed initiated but potentially partially disabled by the virus.

(See Drawing 1.)

TLR7 is constitutively expressed in the small intestines and colon, and present in endosomes in immune cells especially dendritic cells and macrophages. Its expression can also be induced in human airway epithelial cells and primary cardiac cells on infection by viruses. Once TLR-7 binds the targeted ss-RNA virus, a series of messages are communicated and amplified in the cell, mediated by attachment of an intermediary protein called MyD88 to TLR7, then a number of signaling pathways are stimulated to produce an innate immune response to the virus. For TLR7 signaling these pathways can lead to production and release of type 1 interferons (IFN-alpha and IFN-beta), which have antiviral activity, in which case the pathways leading to stimulation of interferon-producing genes include an intermediary signaling protein called TRAF (TRAF3 or TRAF6). Alternatively TLR/MyD88 innate immune responses can trigger a more complex signaling pathway, which also passes through TRAF6, leading to activation of the transcription factor Nf-Kb. In turn, Nf-Kb turns on production and release of pro-inflammatory cytokines including interleukin-6 (IL-6) and IL-12. TLR-8 signaling also uses TRAF3 as a pathway component leading to stimulation of production of type-1 interferons. These complex reactions constitute a major component of the innate immune response that can have a direct antiviral effect to dampen the degree of infection plus lead to presentation of viral antigens needed for the longer term adaptive immune response.

There is evidence (Li et al., 2016) that the papain-like protease (PLPro) produced by the SARS coronavirus as an initial step in replication after the virus has infected a cell can disable TRAF3 and TRAF6 by removing the ubiquitin chains on these signaling proteins at a specific site (Lys63). This results in a reduction in type 1 interferon production by the host organism. Furthermore, another innate immune system pathway that responds to single stranded RNA viruses, mediated by retinoic acid inducible gene 1 (RIG-1), also can be disabled by the SARS virus. The nucleocapsid protein (N) of SARS was shown (Hu et al., 2017) to bind to a motif of the protein TRIM25 which normally activates RIG-1 after RIG-1 recognizes its RNA PAMP. The TRIM25-RIG-1 reaction is also mediated by ubiquitin modification. The SARS N protein blocks this interaction, inhibiting the RIG-1 pathway which would otherwise lead to interferon production.

Based on this potential blockage of type 1 interferon production by coronaviruses, assuming the same mechanisms hold true with the SARS COV-2 virus, the administration of interferon alpha or interferon beta as a prophylactic or early intervention for COVID-19, as suggested above with respect to mimicking bats' always-on interferon adaptation, could be reinforced as a prophylactic or therapeutic strategy. There have been anecdotal reports of beta interferon having a positive effect in COVID-19 patients if administered early upon infection.

The Lectin Pathway and Complement System

Another set of pathways involved in the innate immune system is the complement system, including the traditional pathway, the alternative pathway, and the lectin pathway. These pathways have different mechanisms of initiation but all converge to a common intermediate in the pathways of complement at the protein complement C3, which is the point at which the downstream complement system gets activated. Of these pathways, one potentially most relevant to COVID-19 is the lectin pathway, in particular the component of that pathway based on initiation by Mannose-Binding Lectin (“MBL”, also known as mannan-binding lectin).

Like the TLRs, the role of mannose-binding lectin (MBL) in the innate immune system is to recognize specific molecular structures (PAMPs) of pathogens. In the case of MBL, it recognizes and binds to specific sugar residues of especially mannose, as well as N-acetylglucosamine (GlcNAc) on the glycoproteins of viruses, bacteria, etc. These sugar residues are generally not common on normal human proteins, but do appear over time on some damaged or diseased cells in the human body, which in this case are called DAMPs (Disease-Associated Molecular Patterns). MBL is produced in the liver and circulates in the bloodstream as a complex with two trypsin-like serine proteases called MBL-associated serine protease-1 (MASP-1) or MASP-2. MBL can also circulate in a complex with a truncated form of MASP-2 which lacks the serine protease activity, called MAP19, or MBL can circulate uncomplexed. When MBL binds to a mannose (and/or GlcNAc) residue on a pathogen, MASP-1 and MASP-2 are activated (MASP-1 is thought to activate MASP-2, although both MASP-1 and MASP-2 can autoactivate), and that triggers a number of downstream events. Activated MASP-2 can generate the complement C3 activating protease, C3 convertase, by cleaving C2 and C4 to form C4b2a, thus activating the rest of the complement cascade. The MBL-pathogen complex (connected at the mannose binding site to pathogen's mannose-containing glycoprotein) can be marked by deposition of complement C4 which acts as a direct opsonin to be recognized by antibodies of the adaptive immune system and eliminated. Following C3 activation by MASP-2, the anaphylatoxins C3a and C5a can be generated, both of which are pro-inflammatory mediators. And the terminal component, Complement 5a-9, can form the Membrane Attack Complex (MAC) that lyses damaged cells or pathogens that had been marked in the opsonization process. (review by Noris and Remuzzi, 2013).

(See Drawing 2.)

MBL has been shown to bind to the SARS virus, through mannose residues on the Spike protein. The binding of MBL to SARS virus appears to sterically hinder the ability of the SARS virus to infect host cells, possibly changing the Spike protein conformation or shielding the S1/S2 or S2 cleavage sites from proteases that would otherwise facilitate fusion. Molecular modeling of SARS COV-2 suggests that its Spike protein contains most of the same glycosylation sites as the SARS spike protein plus as many as four new glycosylation sites. Therefore, it is likely that MBL binds to SARS COV-2 as well, perhaps to a different degree or different or additional site(s). The lectin pathway, specifically MBL, may be a key early antiviral response mechanism to fight off COVID-19, potentially in individuals who have been infected but are asymptomatic or have mild disease.

MBL is encoded by the mbl2 gene. There are several known mutations or single nucleotide polymorphisms in the promoter and coding regions of the mbl2 gene that result in no or reduced levels of MBL in circulation (Garcia-Laordin et al., 2008). Individuals with these insufficient levels of MBL are significantly more prone to severe infections, including pneumonia and other lung infections, HIV, and respiratory infections in children, and to impaired lung function in cystic fibrosis. Ip et al. (2005) analyzed more than 500 patients who had SARS vs. over 1000 controls and found that individuals with low-MBL mbl2 polymorphisms were over-represented in the SARS group, and that SARS patients had lower average levels of MBL protein in blood vs. the control group. The prevalence of MBL deficiency due to mbl2 polymorphisms is estimated to be 5-10% in world populations, with a higher incidence in individuals from sub-Sahara Africa and their descendants. Assuming MBL is a key player in the innate immune system antiviral defense against COVID-19, like in SARS, then individuals with these mbl2 polymorphisms may account for some of the cases of higher susceptibility, severe morbidity, or death among COVID-19 patients, and/or may account for some of the incidence of symptomatic or severe cases in otherwise healthy younger patients. Genetic testing of individuals for these mbl2 polymorphisms should be considered for prescreening for the potential of a more severe respiratory infection from SARS COV-2. In addition, MBL levels in blood should be measured in COVID-19 patients as soon as possible after positive diagnosis to assess the potential for more severe infections in individuals with low or no serum MBL protein levels.

On the other hand, there are individuals with above normal levels of MBL circulating in blood. Such cases are often associated with the “DAMP” side of the lectin system. As part of the complement system attack against pathogen infected cells, MBL-marked cells are targeted for destruction by the immune system. If normal human cells or normal glycoproteins are modified through a disease process to aberrantly have more sugar residues such as mannose or N-acetylglucosamine, they can be marked for destruction as well. One major disease that appears to have an association with MBL is diabetes. MBL levels are elevated in Type 1 diabetic patients, especially those with diabetic nephrology and diabetic retinopathy. In addition, levels of MASP-1 and MASP2 are elevated in diabetics and correlate with diabetic control (Jenny et al., 2015). In diabetes, glucose is not fully utilized by cells in the body, either as a result of insufficient production of insulin by the pancreas or reduced ability by cells to take up glucose. As a result, excess sugar builds up in the bloodstream and is deposited on proteins as additional glycosylation. One protein on which excess sugar is deposited is hemoglobin, which carries oxygen through the bloodstream in red blood cells and removes CO2. Diabetic control is often measured by the amount of glycosylation of a hemoglobin type called Hemoglobin A1c, for which measurement of incorporated sugars rises over time in uncontrolled diabetics. There are other hemoglobin types besides A1c. Some hemoglobin has been shown to contain excess mannose residues, and/or are modified with N-Acetylglucosamine, which can be recognized by MBL. With elevated MBL levels in diabetics, the hemoglobin with aberrant mannose levels may be more rapidly tagged for destruction by the complement system. Normally, red blood cells containing the hemoglobin last for about 3-4 months before they are naturally degraded by macrophages, which occurs in the spleen or liver. One of the byproducts of hemoglobin degradation is bilirubin. Red blood cells with hemoglobin are gradually replenished from hematopoietic stem cells by stimulation with erythropoietin.

MBL response to a PAMP is an acute phase reaction following infection. As MBL is bound to the pathogen, levels of free MBL in the bloodstream are initially reduced and expression of additional MBL complex is upregulated. As virus titers increase due to viral replication, presumably more MBL is produced as well. If the additional MBL is targeting both SARS COV-2 and DAMPs such as heavily glycosylated hemoglobin and/or red blood cells, then both positive and negative effects of the MBL directed innate immune response may be expected.

First, on the positive side, with the TLR-7 mediated innate immune response potentially disabled by SARS COV-2 to prevent or limit type 1 interferon production, MBL may be the key to early control of systemic COVID-19 infection by the innate immune system. MBL bound to the SARS virus prevented infection of cell lines in in vitro studies, apparently by blocking the cell fusion step, possibly by hindering the binding of the activating serine protease (e.g., TMPRSS2). MBL's opsonization response may also be the stimulus for the adaptive immune system to kick in with an eventual neutralizing antibody response. Complement C4, the complement of the lectin pathway deposited on pathogens to stimulate their destruction, has been identified deposited on SARS virus in studies (Ip et al., 2005). Therefore, the early response by MBL should be allowed or facilitated, not blocked.

On the negative side, in cases of more severe infections or in patients in which there is a predisposing condition involving DAMPs recognized by MBL, the outcome may turn to an adverse morbidity. For example, in diabetics, if aberrant glycosylated hemoglobin is cleared more rapidly, and replenishment from hematopoietic stem cells can't keep up, hemoglobin levels in patients may drop. This would lead to a reduction in both oxygen supply to tissues and removal of waste CO2. Many severe COVID-19 patients have been reported to have low oxygen saturation in the blood and yet apparently intact functioning lungs not characteristic of ARDS. A reduction in hemoglobin levels could explain these symptoms. Further supporting this possibility, hemoglobin levels as low as 50% of normal have been reported in severe COVID-19 patients, and elevated bilirubin levels are being reported in individuals. In a study (Richardson et al., 2020) of 5700 hospitalized COVID-19 patients in New York, average ferritin levels, a marker for anemia or low red blood cells or excess destruction of red blood cells, were elevated. LDH was also elevated, a sign of low oxygen levels as pyruvate is converted to lactose. A common symptom of COVID-19 is patients gasping for air as if they did not have enough oxygen, and feeling tired or lethargic, which also could result from low oxygenation. However, attempts to re-oxygenate patients through the lungs via mechanical ventilation has not been very successful. (The study of 5700 hospitalized COVID-19 patients in New York reported a mortality rate of 88% in the subset of those patients who were put on ventilators.) These factors suggest the lack of oxygenation in patients was mainly due to an internal systemic dysregulation rather than a respiratory infection-induced blockage of the lungs. Furthermore, it has been widely reported that diabetes, as well as obesity, often considered a prediabetes condition, are major risk factors for severe outcome in COVID-19, again making a connection between MBL/MASP-2 activation and degradation of red blood cells and O₂ carrying hemoglobin.

As a further tie to adverse events in COVID-19 infection, the lectin pathway, which is a primitive immune response mechanism against pathogens found in many lower organisms, has activity in promoting blood coagulation as well as the innate immune response. There is speculation that lower organisms used the lectin pathway to coat pathogens with fibrin, a component of blood clots, as another means of immobilizing the pathogen and limiting the spread of infection. It has been shown that the serine protease MASP-2 can directly cleave prothrombin to generate thrombin, which in turn converts fibrinogen to fibrin, contributing to coagulation of blood. In addition, MASP-1 can directly cleave fibrinogen to form fibrin, and can activate Factor VIII, a protein in the coagulation cascade. These mechanisms lead to fibrous fibrin deposition on blood clots and on damaged tissues or cells, which in turn could lead to such adverse events as deep vein thrombosis, myocardial infarction, disseminated intravascular coagulation, and conditions similar to manifestations of Kawasaki disease. These are all conditions that have been reported in some more severe COViD-19 patients. The potential continuous stimulation of clot formation and fibrin deposition may lead to countervailing attempts by the body to dissolve the clots, leading to breakdown of clot components as well. Levels of D-dimer, a breakdown product of fibrin clots, are elevated on average in hospitalized COVID-19 patients, and correlate with higher probability of death. Other coagulation-related lab findings in COVID-19 patients include thrombocytopenia and prolonged prothrombin-time.

The following are some reports of links between coagulation system disorders or conditions and the lectin pathway/MBL/MASP-1/MASP-2:

“Simultaneous Activation of Complement and Coagulation by MBL-Associated Serine Protease 2” [MASP-2], Krarup, A. et al., PLosONE 7:e623 (2007).

“Activation of mannan-binding lectin-associated serine proteases leads to generation of a fibrin clot”, Gulla, K. et al., Brit. Soc. Immunology 129:482-495 (2009).

“Plasma levels of mannose-binding lectin and future risk of venous thromboembolism”, Liang et al., J. Thromb. Haemost., 10:1661-1669 (2019).

“MASP-1 Induced Clotting—The First Model of Prothrombin Activation by MASP-1”, Jenny et al., PLOS One, pp 1-13, (Dec. 8, 2015).

The following paper discusses the role of MASP-1 in dissolution of clots as well:

“MASP-1 of the complement system alters fibrinolytic behavior of blood clots”, Jenny et al., Mol. Immunol. 114:1-9 (2019)

(See Drawing 3.)

The constant stimulation of the MBL/lectin pathway upon increasing titers of SARS COV-2 during infection may result in an over-activation of the coagulation system by the presence of increasing levels of MBL-associated MASP-1 and MASP-2. The MASP-1 and MASP-2 induced fibrin deposits can then continue to grow into larger clots raising the potential for adverse cardiovascular events. Furthermore, since ACE2 is expressed in the endothelial layers of blood vessels, MBL/MASP1/2 attached to the virus at sites of infection in the blood vessels could lead to microclots on the vessels such as seen in disseminated intravascular coagulation (DIC), as well as vasculitis. This condition manifests itself as red splotches or rashes visible on the skin. It can also be seen in discoloration of the extremities such as toes and fingers, along with inflammation. Similar symptoms can be seen in Kawasaki's Disease and/or Heinoch-Schoenlein Purpura (HSP), both of which have been linked with infectious diseases.

In April and early May 2020, there were increasing reports of COVID-19 positive patients exhibiting symptoms similar to those described above, especially in children. The MBL-based coagulation activation could be the cause of these symptoms. A further complicating factor for this syndrome may be these patients having defective components of the antithrombotic control mechanisms that would normally limit the extent of fibrin deposition and clot formation. One such defect may be Factor V Leiden, an inherited mutation in the Factor V gene that reduces normal Factor V's ability to feed back and inhibit the production of thrombin from prothrombin, thereby stopping further clot formation. Specifically, Protein C, a natural coagulation inhibitor, is unable to bind to Factor V due to the mutation, so that Factor V fails to inhibit the prothrombin to thrombin step. Factor V Leiden polymorphism is found in about 5% of Caucasians in North America, and potentially higher incidence in Europeans. It is rare in other ethnic groups or races. Some of the cases of the “Kawasaki-like” syndrome being reported in otherwise healthy children with COVID-19 may be a result of Factor V Leiden mutations. A genetic test is available for Factor V Leiden mutation and may be a valuable screening tool for newly diagnosed COVID-19 patients.

Many of the reports of the cardiovascular complications from COVID-19 are in patients that have been infected for a period of time, such as a week or more, suggesting that the steps leading to the cardiovascular complications are fairly gradual. This means that there is time after infection for therapeutic intervention. It also means that if the initial symptoms of COVID-19 (fever, for example) resolve quickly, there still may be a risk of progressive deterioration in non-direct viral infection complications such as hypercoagulability, and intervention still may be warranted.

Direct treatment for these coagulation complications after they occur could be by administering anti-thrombin plus heparin, both of which are approved medications. Anti-thrombin binds to thrombin and to Factor Xa, thus inhibiting fibrin deposition. Heparin enhances the binding affinity of anti-thrombin to its targets. Other forms of anticoagulant therapy may be warranted, but given the varied dynamics of clot formation and fibrin dissolution, optimal agents and doses would need to be determined.

An inhibitor of MASP-1 and/or MASP-2 could be a valuable therapeutic option for limiting the coagulation cascade. An inhibitor of MASP-2 (antibody to MASP-2) is in Phase III clinical trials for treating hematopoietic stem cell transplant-related thrombotic microangiopathy (TMA). TMA is a disease or condition in which blood clots form in small blood vessels or capillaries, leading to damaged epithelium, stopped or reduced blood flow, and deformation and bursting of red blood cells. The same antibody drug is also in Phase III trials for treating Hemolytic Uremic Syndrome (HUS), which usually occurs in children in response to an infection or bacterial toxin. HUS is also characterized by endothelial damage especially in the kidney, clotting, and burst red blood cells. These conditions seem to correspond to many of the adverse cardiovascular and coagulation symptoms seen in severe COVID-19 patients.

An inhibitor of Complement C3 is in Phase III clinical trials for treating paroxysmal nocturnal hemoglobinuria, which, in addition to its primary symptom of destruction of red blood cells due to excessive complement activity, is characterized by a high incidence of clot formation. Therefore, this agent could also have a dual role of limiting some or many of the coagulation-related adverse effects of COVID-19, as well as potentially limiting the adverse effects of low oxygen supply and reduced hemoglobin/red blood cell levels from excess or aberrant complement-mediated damage in subsets of patients with COVID-19, as discussed above. However, since C3 falls lower in the complement cascade than MASP-1/MASP-2, and because C3 inhibition would be less likely to have a direct effect on the coagulation activation component, an inhibitor of MASP-1/MASP-2 would likely be a superior choice in treating COVID-19.

One well known condition following a heart attack or other cardiovascular event is reperfusion injury. As oxygen and blood flow is restored, serious complications can continue as the damaged cells are removed and destroyed by the complement system and other immune cells. There have been some reports of COVID-19 patients having adverse cardiovascular events after apparent recovery from the viral infection, which could potentially be related to this condition. Therefore, treatment with MASP-2 or C3 inhibitors or otherwise blocking the MBL pathway following infection and onset of symptoms could still be beneficial, and perhaps even after clearance of the SAR COV-2 infection by a patient, as suggested by the following study.

In a study with transgenic mice expressing human MBL (hu mbl2 knock-in) (Jordan et al, 2001, see also Pavlov et al., 2015), an antibody that blocked MBL administered to mice after an induced myocardial infarction and initial reperfusion showed the following: preserved myocardial function, reduced infarct size, prevented fibrin deposition within the myocardium, and prevented occlusive arterial thrombogenesis. These positive effects were all seen with MBL antibody treatment after the cardiovascular event, suggesting that an MBL/MASP-2 inhibition after the onset of SARS COV-2 infection might still be beneficial to resolve symptoms.

As further possible suggestion for the involvement of the lectin pathway and/or specific glycoprotein sugar residues in COVID-19 susceptibility and morbidity, in a case study in China (Zhou et al., 2020), significantly higher risks of infection and death were found to be associated with blood type A patients, and significantly lower respective rates were observed in blood type O patients. People with blood type A have the A antigen expressed on the surface of their red blood cells. “A” antigen is formed by first the enzyme fucosyltransferase adding fucose sugar residues to surface proteins on cells to form the H antigen, then the enzyme glycosyltransferase adds N-acetylglucosamine moieties on the deposited fucose (the glycoprotein is now called the “A” antigen). MBL binds N-acetylglucosamine, in addition to mannose. People with “O” blood type do not have the A antigen on their blood cells but have circulating anti-A antibodies, which are not present in Type A blood patients. In a study with SARS, Guillon, et al. (2008) showed that anti-A antibodies specifically inhibited binding of the spike protein of SARS to ACE2 receptor-expressing cell lines, suggesting a possible explanation for observed lower infection rates in individuals with type O blood, and again suggesting a possible positive role of MBL itself with respect to SARS COV-2.

If this disease progression is correct, then potential therapeutic options could include the following:
1. Using a diagnostic test that is accurate and detects early infection and/or viral titers in patients, taking no intervention with respect to this mechanism for a period after infection (or at below a certain threshold of viral titer) that would allow the lectin pathway/MBL to develop an initial innate immune response to SARS COV-2. Based on the general guidance that symptoms do not develop until about 5 days after initial infection, no intervention may extend for a period of several days into the infection. Thereafter, the treatment options might be:
   An inhibitor of the serine proteases MASP-1 or MASP-2 that would stop the complement cascade at the beginning of the process. A natural inhibitor of MASP-1 is sunflower trypsin inhibitor (SFTI), found in the seeds of sunflowers, and other Bowman-Birk inhibitors. An antibody against MASP-2 (narsoplimab; OMS-721) is in Phase 3 trials for treatment of hematopoietic stem cell transplant-related thrombotic microangiopathy, a disease in which endothelial dysfunction leads to microangiopathic hemolytic anemia, platelet activation, and formation of platelet-rich thrombi. Since MASP-2 is the only enzyme of these two shown to have the ability to autoactivate the complement cascade, MASP-2 blocker would be the preferred target of these two.
   An inhibitor of complement C3 that would stop the complement cascade at the junction of the three different complement pathways. A C3 inhibitor (Pegcetaclopan) is in Phase 3 trials for treating proximal nocturnal hemoglobinuria (PNH), a rare genetic disease in which hemoglobin levels are depressed.
   An inhibitor of complement C5, which is one of the final components in the complement cascade and is associated with a pro-inflammatory response. A C5 inhibitor (eculizumab, or Soliris) is a marketed drug for treating PNH. This approach may be too late in the complement cascade to counteract some of the potential mechanisms discussed above, but could reduce symptoms of excessive inflammatory response.
   An inhibitor of the receptor for complement C5a (Avacopan; CCX-168) is in Phase III trials for treating ANCA associated vasculitis, which is an inflammation of the small blood vessels in the body caused by anti-neutrophilic cytoplasmic autoantibodies. Blocking the C5a receptor could reduce some of the pro-inflammatory consequences of complement system activation but as a late cycle mediator may not be as relevant as an earlier stage inhibitor of the MBL pathway.
2. Administering potential blockers of the interaction between MBL and its mannose or N-acetylglucosamine targets on glycoproteins such that these mimics of the MBL target are occupied without activating MASP-1 or MASP-2. These might include D-mannose and other small sugar derivatives that bind to MBL competitively with its glycoprotein targets.
3. Administering mimics of the mannose-binding function of MBL that cannot complex with MASP-1 or MASP-2 to activate the complement system. Specifically this might include mannose lectins that were mentioned above as potential treatments or prophylactics by oral consumption to prevent SARS COV-2 from entering through the small intestines, such as banana lectin, or griffithsin, or lower molecular weight fragments of such lectins that would retain the mannose binding capability, or other similarly functioning molecules. These lectins would be administered systemically rather than (or in addition to) the proposed oral consumption route. For example, griffithsin, a 12.7 kD protein isolated from a red algae with strong mannose-binding properties, has demonstrated potent in vitro and in vivo antiviral activity against SARS virus (O'Keefe et al., 2010). The protein is being produced in genetically engineered tobacco plants for clinical testing as a microbiocide against HIV transmission. Derivatives of griffithsin with lower molecular weight and/or altered pharmacologic or pharmacodynamics properties have been produced including grifonin-1 (Micewicz et al., 2010). Similarly, an altered form of banana lectin, called H84T, has been developed that eliminates a mitogenic activity of banana lectin (Swanson et al., 2015) while preserving its antiviral properties, specifically against Ebola (Coves-Datson, 2019). Another option might be modified MBL that cannot complex with MASP-1/MASP-2. A recombinant MBL was previously produced but apparently discontinued in clinical studies. In a preclinical study, mice administered high dose rMBL (7× that normally in human serum) survived otherwise fatal challenge with Ebola virus and became immune to virus re-challenge (Michelow et al., 2011). Whether this form of MBL became complexed with MASP-1/MASP-2 after it was administered, or would be in humans, is uncertain.
4. For non-drug medical intervention, steps to replenish functional hemoglobin and red blood cells should be undertaken as soon as soon as possible and be sustained. This could include administering erythropoietin (marketed drug) and/or hematopoietic stem cells. Erythropoietin may have a lag of several days before a significant increase in mature red blood cells is seen. Blood transfusions could also be used as an immediate step, especially in severe cases of reduced hemoglobin levels.

One further possibility that should be explored experimentally is whether MASP-1 or MASP-2, both trypsin-like serine proteases, can cleave the SARS COV-2 spike protein at S1/S2 and/or the S2 site, as has been shown for TMPRSS2, another trypsin-like serine protease. In such case, MBL could bind SARS COV-2 in a position to be activated by MASP-1 or MASP-2. This could result in activated SAR COV-2 circulating in the bloodstream, or SARS COV-2 attaching to the ACE2 receptor in a pre-activated site for entry into a host cell without the need for TMPRSS2 co-expression. If this were the case, it is likely that a MASP-1 and/or MASP-2 inhibitor would be a direct treatment for SARS COV-2 infection, not just COVID-19 secondary effects.

In this regard, again if this hypothetical activation phenomena as mentioned above is demonstrated, an inhibitor of MASP-2 (narsoplimab) in Phase III clinical trials for other indications could be a potential treatment option to block the serine protease mediated activation of SARS COV-2. With respect to blocking MASP-1, and potentially more generally blocking other serine proteases that may be able to activate SARS COV-2, administration of plant-based serine protease inhibitors (serpins) or derivatives thereof may potentially be effective antiviral treatments for SARS COV-2. Slightly modified forms of sunflower trypsin inhibitor-1 (SFTI) were shown (Heja et al., 2012) to potently inhibit the activity of both MASP-1 and MASP-2 (with higher inhibitory activity against MASP-1). SFTI-1 is a 14-mer cyclic peptide found in relatively high levels in sunflower seeds. There have been numerous efforts to use SFTI-1 as a scaffold to synthesize novel serine protease inhibitors. If MASP-1/MASP-2 does activate SARS-COV-2 for cell fusion similar to the reported role of the serine protease TMPRSS2, and SFTI-1 or other plant serpins can inhibit these serine proteases, then they may be able block SARS COV-2 infection. Even if MASP-1/MASP-2 are not involved, SFTI-1/plant serpins may be useful for blocking TMPRSS2 or furin or other serine protease activators of SARS COV-2. SFTI-1 could potentially be delivered orally via consumption of sunflower seeds in an amount and on a time schedule of dosing that one skilled in the art could determine. Since sunflower seeds are eaten by humans as a common food source, adverse events are unlikely. The sunflower seeds/SFTI-1 or other serpins could limit the infectivity of SARS COV-2 in the gut and/or may be absorbed into the bloodstream via the gut to act systemically. Consumption of sunflower seeds could be combined with consumption of mannose lectin sources such as bananas, as previously discussed. Banana lectin could bind to the SARS COV-2 spike protein and limit infectivity through a different mechanism of preventing binding or fusion with a host cell by steric hindrance or other mechanism. Alternatively, SFTI-1/derivatives/alternative plant serpins and/or banana lectins/derivatives/alternative mannose lectin sources could be formulated as drugs to be administered systemically via injection, infusion, or intravenously.

A recent related but slightly different mechanism for MBL and SARS COV-2 interaction that has been proposed (Gao et al, unreviewed preprint 2020) is that MBL can bind to the nucleocapsid glycosylated protein (N) of SARS COV-2, either separately from, or in addition to, its binding to the spike protein. The binding of N and MBL activates MASP-2, and then SARS COV-2 infects the host cell and replicates. When the new viral particles are released, N proteins are also released into the bloodstream, which are bound by more MBL, activating MASP-2, and triggering further complement activation. This creates a positive feedback loop from infection that multiplies the degree of complement activation and resultant pro-inflammatory response, leading to worsening secondary symptoms in COVID-19 patients. If this mechanism is correct, treatment with a blocker of MASP-2 would be a preferred therapeutic approach.

(See Drawing 4.)

Implications of MBL-Associated Effects for Vaccine Development

Based on the assumption that molecular structures (PAMPS) on the SARS COV-2 virus (Spike and/or Nucleocapsid proteins, including their glycosylation sites) may be inducing lectin pathway-mediated immunological responses plus adverse coagulation system effects, care must be taken to ensure that any vaccine developed against SARS COV-2 not stimulate any adverse coagulation system effects after administration. Coagulation markers should be monitored in those individuals participating in clinical trials of vaccine candidates, in tandem with the usual immunological goals of looking for neutralizing antibodies and/or T-cell responses. Furthermore, the impact on more vulnerable population subsets, potentially including those individuals who have the Factor V Leiden mutation or other relevant polymorphisms or risk factors noted herein, should be evaluated during vaccine safety testing.

### REFERENCES CITED

The entirety of the references cited are hereby relied upon and incorporated by reference herein.
Hamming I, Timens W, Bulithuis M L C, Lely A T, Navis G J, van Goor H. (2004). Tissue distribution of ACE2 proteins, the functional receptor for SARS coronavirus. A first step in understanding SARS pathogenesis. J Pathol 2004; 203: 631-637.
Ziegler et al. (2020). SARS-CoV-2 Receptor ACE2 Is an Interferon-Stimulated Gene in Human Airway Epithelial Cells and Is Detected in Specific Cell Subsets across Tissues. Cell (2020), 181:1-20.
Chen, C; Gao, G; Xu, Y; Pu, L; Wang, Q; Wang, L; et al. (12 others) (2020). SARS-CoV-2-Positive Sputum and Feces After Conversion of Pharyngeal Samples in Patients With COVID-19. Annals of Internal Medicine; Annals.org pub Mar. 30, 2020. pp. 1-3.
Fuqua J L, Wanga V, Palmer K E. (2015). Improving the large scale purification of the HIV microbicide, griffithsin. BMC Biotechnol. 2015 Feb. 22; 15(1):12. pp. 1-10.
Alam A, Jiang L, Kittleson G A, Steadman K D, Nandi S., Fuqua J L, Palmer K E, Tuse' D, McDonald K A (2018). Technoeconomic modeling of plant-based griffithsin manufacturing. Front Bioeng Biotechnol. 2018 Jul. 24; 6:102.
Li D, Jin M, Bao P, Zhao W, Zhang S. (2020) Clinical characteristics and results of semen tests among men with coronavirus disease 2019. JAMA Netw Open. 2020; 3(5):e208292. pp 1-3.
Lin B, Ferguson C, White J T, Wang S, Vessella R, True L D, Hood L, and Nelson P S. (1999). Prostate localized and androgen-regulated expression of the membrane-bound serine protease TMPRSS2. Cancer Research 59, 4180-4184, Sep. 1, 1999.
Zhou F, Yu T, Du R, Fan G, Liu Y, Liu, Z, Zhang J, Wang Y, Song B, Gu X, Guan L, Wei Y, Li H, Wu X, Xu J, Tu S, Zhang Y, Chen H, Cao B. (2020). Clinical course and risk factors for mortality of adult inpatients with Covid-19 in Wuhan, China: a retrospective cohort study. Lancet 2020; 395: 1054-62 Published Online Mar. 9, 2020.
Swanson, M. D., Winter, H. C., Goldstein, I. J., and Markovitz, D. M. (2010). A lectin isolated from bananas is a potent inhibitor of HIV replication. J. Biol. Chem. 285, 8646-8655.
Hopper J T S, Ambrose S, Grant O C, Krumm S A, Allison T M, Degiacomi M T, Tully M D, Pritchard L K, Ozorowski G, Ward A B, Crispin M, Doores K J, Woods R J, Benesch J L P, Robinson C V, Struwe W B. (2017). The Tetrameric Plant Lectin BanLec Neutralizes HIV through Bidentate Binding to Specific Viral Glycans. Structure. 2017 May 2; 25 (5):773-782.e5.
Covés-Datson E M, Dyall J, DeWald L E, King S R, Dube D, Legendre M, Nelson E, Drews K C, Gross R, Gerhardt D M, Torzewski L, Postnikova E, Liang J Y, Ban B, Shetty J, Hensley L E, Jahrling P B, Olinger G G Jr, White J M, Markovitz D M. (2019). Inhibition of Ebola Virus by a Molecularly Engineered Banana Lectin. PLoS Negl Trop Dis. 2019 Jul. 29; 13 (7):e0007595.
KEYAERTS, E; VUGEN, L; PANNECOUQUE, C; Van Damme, E; PEUMANS, W; EGBERINK, H; BALZARINI, J: and VAN RANST, M. (2007). Plant lectins are potent inhibitors of coronaviruses by interfering with two targets in the viral replication cycle. (2007) ANTIVIRAL RESEARCH. 75(3). p. 179-187.
Li S W, Wang C Y, Jou Y J, Huang S H, Hsiao L H, Wan L, Lin Y J, Kung S H, Lin C W. (2016). SARS Coronavirus Papain-Like Protease Inhibits the TLR7 Signaling Pathway through Removing Lys63-Linked Polyubiquitination of TRAF3 and TRAF6. Int J Mol Sci. 2016 May 5; 17(5):678.
Hu Y, Li W, Gao T, Cui Y, Jin Y, Li P, Ma Q, Liu X, Cao C. (2017). The severe respiratory syndrome coronavirus nucleocapsid inhibits Type I interferon production by interfering with TRIM25-mediated RIG-I ubiquitination. J Virol. 2017 Mar. 29; 91(8):e02143-16.
Noris M, and Remuzzi G. (2013). Overview of complement activation and regulation. Semin Nephrol. 2013 November; 33(6):479-92.
Garcia-Laorden M I, Sole-Violan J, Rodriguez de Castro F, Aspa J, Briones M L, Garcia-Saavedra A, Rajas O, Blanquer J, Caballero-Hidalgo A, Marcos-Ramos J A, Hemandez-Lopez J, Rodriguez-Gallego C. (2008). Mannose-binding lectin and mannose-binding lectin-associated serine protease 2 in susceptibility, severity, and outcome of pneumonia in adults. J Allergy Clin Immunol. 2008 August; 122(2):368-74, 374.e1-2.
Ip W K E, Chan K H, Law H K W, Tso G H W, Kong E K P, Wong W H S, To Y F, Yung R W H, Chow E Y, Au K L, Chan E Y T, Lim W, Jensenius J C, Turner M W, Pereis J S M, and Lau Y L. (2005). Mannose-binding lectin in severe acute respiratory syndrome coronavirus infection. J Infectious Disease. 2005 May 15; 191(10):1697-704. Epub 2005 Apr. 11.
Jenny L, Ajjan R, King R, Thiel S, Schroeder V. (2015). Plasma levels of mannan-binding lectin-associated serine proteases MASP-1 and MASP-2 are elevated in type 1 diabetes and correlate with glycaemic control. Clin Exp Immunol. 2015 May; 180(2):227-32
Richardson S, MD, MPH, Hirsch J S, MD, MA, MSB, Narasimhan M, DO, Crawford J M, MD, PhD, Mcginn T, MD, MPH, Davidson K W, PhD, MASc, and the Northwell COVID-19 Research Consortium. (2020). Presenting characteristics, comorbidities, and outcomes among 5700 patients hospitalized with COVID-19 in the New York City area. JAMA. 2020 Apr. 22: e206775.
Krarup A, Wallis R, Presanis J S, Gál P, Sim R B. (2007). Simultaneous activation of complement and coagulation by MBL-associated serine protease 2. PLoS One. 2007 Jul. 18; 2(7):e623.
Gulla K C, Gupta K, Krarup A, Gal P, Schwaeble W J, Sim R B, O'Connor C D, Hajela K. (2010). Activation of mannan-binding lectin-associated serine proteases leads to generation of a fibrin clot. Immunology. 2010 April; 129 (4):482-95.
Liang R A, Høiland I I, Ueland T, Aukrust P, Snir O, Hindberg K, Braekkan S K, Garred P, Mollnes T E, Hansen J B. (2019). Plasma levels of mannose-binding lectin and future risk of venous thromboembolism. J Thromb Haemost. 2019 October; 17 (10):1661-1669.
Jenny L, Dobó J, Gál P, Schroeder V (2015). MASP-1 of the complement system promotes clotting via prothrombin activation. Mol Immunol. 2015 June; 65(2):398-405.
Jenny L, Dobó J, Gil P, Schroeder V (2015). MASP-1 Induced Clotting—The First Model of Prothrombin Activation by MASP-1. PLoS ONE 10 (12): e0144633.
Jenny L, Noser D, Larsen J B, Dobó J, Gál P, Pál G, Schroeder V. (2019). MASP-1 of the complement system alters fibrinolytic behaviour of blood clots. Mol Immunol. 2019 October; 114:1-9.
Jordan J. E., Montalto M. C., Stahl G. L. (2001). Inhibition of mannose-binding lectin reduces postischemic myocardial reperfusion injury. Circulation. 2001; 104:1413-1418.
Pavlov, V I; Tan, Y S; McClure, E E; La Bonte, L R; Zou, C; Gorsuch, W B; and Stahl, G L. (2015). Human Mannose-Binding Lectin Inhibitor Prevents Myocardial Injury and Arterial Thrombogenesis in a Novel Animal Model. American Journal of Pathology, Vol. 185, No. 2, February 2015.
Zhou P, Tachedjian M, Wynne J W, Boyd V, Cui J, Smith I, Cowled C, Ng J H J, Mok L, Michalski W P, Mendenhall I H, Tachedjian G, Wang L-F and Baker M. (2015) Contraction of the type I IFN locus and unusual constitutive expression of IFN-alpha in bats. 2696-2701 PNAS, Mar. 8, 2015, vol. 113, no. 10.
Guillon, P; Clément, M; Sébille, V; Rivain, J; Chou, C; Ruvoën-Clouet, N; Le Pendu, J. (2008). Inhibition of the interaction between the SARS-CoV spike protein and its cellular receptor by anti-histo-blood group antibodies. Glycobiology 2008 December; 18(12):1085-93.
O'Keefe B R, Giomarelli B, Barnard D L, Shenoy S R, Chan P K S, McMahon J B, Palmer K E, Barnett B W, Meyerholz D K, Wolford-Lenane C L, McCray P B (2010). Broad-spectrum in vitro activity and in vivo efficacy of the antiviral protein griffithsin against emerging viruses of the family Coronaviridae. J. Virol. 2010 March; 84(5):2511-21.
Micewicz E D, Cole A L, Jung C-L, Luong H, Phillips M L, Pratikhya P, Sharma S, Waring A J, Cole A M, Ruchala P. (2010). Grifonin-1: a small HIV-1 entry inhibitor derived from the algal lectin, Griffithsin. PLoS ONE. 2010 Dec. 16; 5(12):e14360.
Covés-Datson E M, Dyall J, DeWald L E, King S R, Dube D, Legendre M, Nelson E, Drews K C, Gross R, Gerhardt D M, Torzewski L, Postnikova E, Liang J Y, Ban B, Shetty J, Hensley L E, Jahrling P B, Olinger G G Jr, White J M, Markovitz D M. (2019). Inhibition of Ebola Virus by a Molecularly Engineered Banana Lectin. PLoS Negl Trop Dis. 2019 Jul. 29; 13(7):e0007595.
Swanson M D, Boudreaux D M, Salmon L, Chugh J, Winter H C, Meagher J L, Andre S, Murphy P V, Oscarson S, Roy R, King S, Kaplan M H, Goldstein U, Tarbet E B, Hurst B L, Smee D F, de la Fuente C, Hoffmann H H, Xue Y, Rice C M, Schols D, Garcia J V, Stuckey J A, Gabius H J, Al-Hashimi H M, Markovitz D M. (2015). Engineering a therapeutic lectin by uncoupling mitogenicity from antiviral activity. Cell. 2015 Oct. 22; 163(3):746-58.
Michelow I C, Lear C, Scully C, Prugar L I, Longley C B, Yantosca L M, Ji X, Karpel M, Brudner M, Takahashi K, Spear G T, Ezekowitz R A, Schmidt E V, Olinger G G (2011). High-dose mannose-binding lectin therapy for Ebola virus infection. J Infect Dis. 2011 Jan. 15; 203(2):175-9.
Heja D, Harmat V, Fodor K, Wilmanns M, DoboJ, Kekesi K A, Zavodszky P, Gal P, and Pal G. (2012). Monospecific Inhibitors Show That Both Mannan-binding Lectin-associated Serine Protease-1 (MASP-1) and -2 Are Essential for Lectin Pathway Activation and Reveal Structural Plasticity of MASP-2. April 2012 Journal of Biological Chemistry 287(24):20290-300.
Gao, T.; Hu, M.; Zhang, X.; et al. (32 others). (2020). Highly pathogenic coronavirus N protein aggravates lung injury by MASP-2-mediated complement over-activation. MedRxiv (preprint). doi: https//doi.org/10.1101/2020.03.29.20041962.

What is claimed:

1. A method for prevention or treatment of COVID-19 by administering to a human a pharmacologically effective dose of a lectin that binds to residues of mannose and/or N-acetylglucosamine that are present on the surface proteins of the SARS COV-2 virus.

2. The method of claim 1 wherein the lectin binds to the spike protein (S) of the SARS COV-2 virus such that (a) the interaction and/or binding of S with or to its target receptor on a human cell is inhibited or (b) the ability of SARS COV-2 to become internalized by or infect its target human cell is partially or fully prevented.

3. The method of claim 2 wherein the target receptor on the human cell is Angiotensin Converting Enzyme-2 (ACE2).

4. The method of claim 1 wherein the lectin is contained in or derived from a vegetable or fruit including but not limited to banana, mango, fig, avocado, jackfruit, pineapple, leek, soybean, or garlic.

5. The method of claim 1 wherein the lectin is banana lectin or a derivative thereof.

6. The method of claim 1 wherein the lectin is contained in or derived from algae including but not limited to those from the genera Griffithsia, Porphyra, Palmeria, Agardhiella, or Gracilaria, or other genera of red algae.

7. The method of claim 1 wherein the lectin is griffithsin or a derivative thereof.

8. The method of claim 1 wherein the lectin is human mannose- (or mannan-) binding lectin (MBL) or a derivative or recombinant form of MBL.

9. The method of claim 8 wherein the MBL is modified such that it does not activate mannose associated serine protease-1 (MASP-1) or mannose associated serine protease-2 (MASP-2).

10. A method for treatment of adverse coagulation dysfunction and/or inflammatory sequelae of COVID-19 infection by administering to a human a pharmacologically affective dose of an agent that acts as an inhibitor of the lectin complement pathway.

11. The method of claim 10 wherein the agent is an inhibitor of mannose associated serine protease-1 (MASP-1) or mannose associated serine protease-2 (MASP-2).

12. The method of claim 11 wherein the inhibitor of MASP-1 or MASP-2 is an antibody directed against MASP-1 or MASP-2, respectively, which reduces or blocks the enzymatic activity of MASP-1 or MASP-2.

13. The method of claim 11 wherein the inhibitor of MASP-1 or MASP-2 is a small molecule or peptide that reduces or blocks the enzymatic activity of MASP-1 or MASP-2.

14. The method of claim 13 wherein the small molecule or peptide inhibitor is sunflower protease inhibitor-1 (SFPI-1), a derivative of SFPI-1, or other Bowman-Birk inhibitor or a derivative thereof.

15. The method of claim 10 wherein the agent is an inhibitor of Complement C3 or Complement C5.

16. The method of claim 10 in which the agent is administered to a human during a time window between (a) the date of onset of initial systemic infection by the SARS COV-2 virus plus a period, for example two days after onset, in which a complement-mediated immune response is initiated in the human, and (b) the date before systemic coagulation dysfunction is manifested in the human, for example being seven days after the onset of systemic infection by SARS COV-2.

17. A method for treatment of coagulation dysfunction or other related adverse events in a human in connection with that human being exposed systemically to the spike protein (S) of SARS COV-2 or to a modification or a derivative, of S, or subcomponent of S that includes the ACE2 receptor binding site of S, or genetic material that encodes for S or a portion thereof, by administering to that human a pharmacologically affective dose of an agent that acts as an inhibitor of the lectin complement pathway.

18. The method of claim 17 wherein the human has been administered a vaccine against COVID-19 in which the spike protein of SARS COV-2, or derivatives or modifications thereof, is present in the vaccine or expressed systemically in the human as a result of being administered the vaccine.

19. A method for identifying humans who are at increased risk of adverse events due to coagulation dysfunction, including but not limited to blood clots, in connection with COVID-19 infection by testing individuals for genetic polymorphisms related to the blood coagulation system, including but not limited to Factor V Leiden.

20. A method for identifying humans who are at increased risk of adverse events due to coagulation dysfunction, including but not limited to blood clots, in connection with the human being administered a vaccine against COVID-19 in which the spike protein of SARS COV-2, or derivatives thereof, is present in the vaccine or expressed in the human as a result of being administered the vaccine, by testing or prescreening individuals for genetic polymorphisms related to the blood coagulation system, including but not limited to Factor V Leiden.
